(19) United States
(12) Patent Application Publication
(10) Pub. No.: US 2023/0158230 A1
(43) Pub. Date: May 25, 2023

(54) INFUSION APPARATUS AND METHOD FOR TESTING EXTRAVASATION

(71) Applicant: Chang Gung University, Taoyuan City (TW)

(72) Inventors: SHIN-YU LIEN, Taoyuan City (TW); CHAO-SUNG LAI, Taoyuan City (TW); YA-JU CHANG, Taoyuan City (TW)

(21) Appl. No.: 17/937,789

(22) Filed: Oct. 4, 2022

(30) Foreign Application Priority Data
Nov. 19, 2021 (TW) 110143106

(51) Int. Cl. A61M 5/168; A61M 5/158

(52) U.S. Cl. CPC A61M 5/16836; A61M 5/158; A61M 5/16804

(57) ABSTRACT

An infusion apparatus and a method for testing extravasation are provided. The infusion apparatus includes a liquid driver, a detection unit, and a controller. The controller controls the liquid driver to be operated, so that an input liquid provided by a infusion supply unit is injected into a living body through a tube and a needle, and a blood return detecting procedure is performed. The blood return detecting procedure includes a stop driving step implemented by controlling the liquid driver to stop operating; and a determining step implemented by controlling the detection unit to test a state of a section of the tube adjacent to the needle to generate a detected signal, and determining whether or not the blood of the living body returns to the tube according to the detected signal.

### CROSS-REFERENCE TO RELATED PATENT APPLICATION

This application claims the benefit of priority to Taiwan Patent Application No. 110143106, filed on Nov. 19, 2021. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an infusion apparatus and a method for testing, and more particularly to an infusion apparatus and a method for testing extravasation.

### BACKGROUND OF THE DISCLOSURE

In a process of intravenous infusion, extravasation often occurs as a result of various circumstances (e.g., a patient receiving intravenous infusion moving and causing a needle to leave the vein of the patient). When the extravasation has occurred, if relevant personnel do not properly deal with the extravasation in a timely manner, liquid that is input will be continuously injected into the tissue around an injection position, and the patient may experience swelling, inflammation, or pain at the point of injection and its peripheral areas. In addition, certain chemotherapeutic agents are corrosive, and if the extravasation occurs and the relevant personnel do not deal with the extravasation involving corrosive chemotherapeutic agents in time, this may cause severe consequences to the patient, such as amputation of the extremity on which the injection position is located.

### SUMMARY OF THE DISCLOSURE

In response to the above-referenced technical inadequacy, the present disclosure provides an infusion apparatus and a method for testing extravasation, primarily improving on a conventional intravenous infusion apparatus (e.g., an IV pump) that does not have an extravasation testing ability. Relevant personnel are required to check whether the extravasation has occurred according to professional experience, and inexperienced personnel may not detect the occurrence of the extravasation.

In one aspect, the present disclosure provides an infusion apparatus. The infusion apparatus includes a liquid driver, a detection unit, and a controller. The liquid driver is connected to at least one infusion supply unit. The liquid driver is connected to one end of a tube, and another end of the tube is connected to a needle. The detection unit is configured to test a state of a section of the tube adjacent to the needle to correspondingly generate a detected signal. The controller is electrically connected to the liquid driver and the detection unit. The controller is configured to control the liquid driver to be operated, so that an input liquid provided by the at least one infusion supply unit is injected into a living body and a blood return detecting procedure is performed by the controller. The blood return detecting procedure includes controlling the liquid driver to stop operating to reduce a hydraulic pressure in the tube; and controlling the detection unit to test the state of the section of the tube adjacent to the needle to generate the detected signal, so as to determine whether or not the blood of the living body returns to the section of the tube adjacent to the needle or not. In response to the blood of the living body not returning to the section of the tube adjacent to the needle, an extravasation is determined to have occurred and a notification data is correspondingly generated. In response to determining that the blood returns to the section of the tube adjacent to the needle, the extravasation is determined to not have occurred and the liquid driver is re-operated.

In another aspect, the present disclosure provides a method for detecting extravasation applied in an infusion apparatus. The infusion apparatus is connected to a tube. One end of the tube is connected to a needle. The method includes stopping injecting an input liquid from at least one infusion supply unit to reduce a hydraulic pressure in the tube; and detecting a state of a section of the tube adjacent to the needle to correspondingly generate a detected signal and determining whether the blood of the living body returns to the section of the tube adjacent to the needle according to the detected signal. In response to determining that the blood does not return to the section of the tube adjacent to the needle, the extravasation is determined to have occurred and a notification data is correspondingly generated. In response to determining that the blood returns to the section of the tube adjacent to the needle, the extravasation is determined to not have occurred and the liquid driver is re-operated.

Therefore, the infusion apparatus and the method for testing extravasation of the present disclosure can help relevant personnel find and solve the extravasation in time. Accordingly, damage to the living body due to extravasation can be effectively prevented.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of an infusion apparatus, an infusion supply unit, a tube, and a needle of the present disclosure;

FIG. 2 is a block diagram of an infusion apparatus according to a first embodiment of the present disclosure;

FIG. 3 is a flowchart of a blood return detecting procedure of the present disclosure;

FIG. 4 is a block diagram of an infusion apparatus according to a second embodiment of the present disclosure;

FIG. 5 is a block diagram of an infusion apparatus according to a third embodiment of the present disclosure;

FIG. 6 is a block diagram of an infusion apparatus according to a fourth embodiment of the present disclosure;

FIG. 7 is a schematic view of an infusion apparatus according to a fifth embodiment of the present disclosure;

FIG. 8 is a schematic view of an infusion apparatus according to a sixth embodiment of the present disclosure;

FIG. 9 is a block diagram of an infusion apparatus according to a seventh embodiment of the present disclosure; and

FIG. 10 is a flowchart of a method for testing extravasation of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

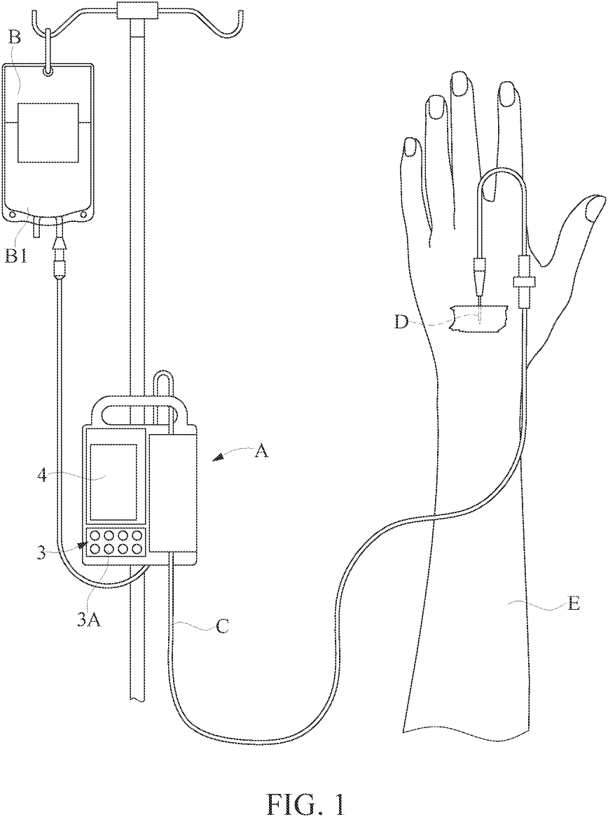

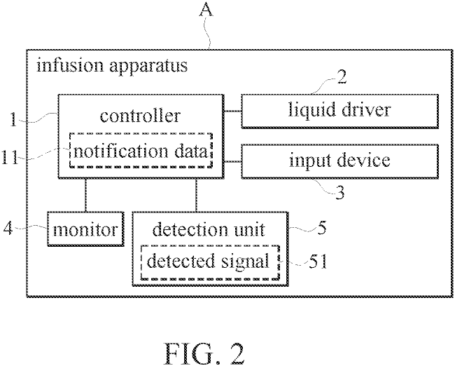

Referring to FIG. 1 and FIG. 2, FIG. 1 is a schematic view of an infusion apparatus, an infusion supply unit, a tube, and a needle of the present disclosure, and FIG. 2 is a block diagram of an infusion apparatus according to a first embodiment of the present disclosure. An infusion apparatus A of the present disclosure includes a controller 1, a liquid driver 2, an input device 3, a monitor 4, and a detection unit 5. It should be noted that, the infusion apparatus A of the present disclosure can be provided without the input device 3 and the monitor 4 being included.

The controller 1 is electrically connected to the liquid driver 2, the liquid driver 2 is connected to an infusion supply unit B (e.g., a drip bag) and one end of a tube C, another end of the tube C is connected to a needle D, and the needle D is configured to be inserted into a blood vessel (e.g., a vein) of a living body E. The controller 1 is configured to control the liquid driver 2 to be operated, so that an input liquid B1 provided by the infusion supply unit B is injected into the living body E through the tube C. The living body E can be, for example, a human being or a pet, and the present disclosure is not limited thereto. The liquid driver 2 can include a liquid driving unit (e.g., a peristaltic pump or a motor), and the controller 1 can control the liquid driver 2 to be operated, so that the input liquid B1 provided by the infusion supply unit B can be injected into the living body E with a stable flow speed and a stable flow amount. More specifically, through a cooperation of components (e.g., the controller 1 and the liquid driver 2), the infusion apparatus A of the present disclosure can achieve the effect of a conventional intravenous infusion pump.

The input device 3 is electrically connected to the controller 1, and the input device 3 can be operated by a user to control the liquid driver 2 through the controller 1. Specifically, the input device 3 can exemplarily include a plurality of physical buttons, and the user can configure the flow speed and the flow amount of the input liquid B1 provided by the infusion supply unit B and injected into the living body E by the infusion apparatus A by pressing a portion of the physical buttons. On the other hand, the user can control the infusion apparatus A to be operated (i.e., turned on) or not operated (i.e., turned off) by pressing another portion of the physical buttons. Naturally, the input device 3 can be provided to not include the physical buttons. In other embodiments, the input device 3 and the monitor 4 can be integrated into a touchscreen, and the user can turn on or turn off the infusion apparatus A, or control the flow speed and amount of the input liquid B1 injected into the living body E by the infusion apparatus A by operating the touchscreen.

In a special application, the input device 3 can be a wired or wireless interface (e.g., a USB interface and Wi-Fi®), and a related electronic apparatus (e.g., a smartphone, a tablet, or a remote server) independent from the infusion apparatus A can transmit related data to the infusion apparatus A in a wired manner or in a wireless manner through the input device 3, so that the infusion apparatus A can be turned on or turned off, or the infusion apparatus A can be controlled to inject the input liquid B1 into the living body E with a predetermined flow speed and a predetermined flow amount.

The monitor 4 is electrically connected to the controller 1. When the user controls the input device 3, the controller 1 can control the monitor 4 to show a flow amount value and a flow speed value, and the user can configure the flow speed and flow amount of the input liquid B1 provided by the infusion supply unit B and injected into the living body E by operating the input device 3 and viewing the monitor 4. It should be noted that, in one embodiment of the present disclosure, the infusion apparatus A does not include the monitor 4, the input device 3 can include a knob, and the user can operate the knob to cause the infusion apparatus A to inject the input liquid B1 into the living body E with different flow speeds and different flow amounts.

The detection unit 5 is configured to test a state of a section of the tube C adjacent to the needle D to correspondingly generate a detected signal 51. The detection unit 5 is configured to be connected to the controller 1 in a wired manner or a wireless manner, the controller 1 can control the detection unit 5 to be operated, the detection unit 5 can transmit the detected signal 51 to the controller 1, and the controller 1 can determine whether or not the blood of the living body E returns the section of the tube C adjacent to the needle D according to the detected signal 51.

In a practical application, the detection unit 5 can be fixed to the section of the tube C adjacent to the needle D through a relevant fixing component. Or, the detection unit 5 can be an assembly independent from the tube C, the detection unit 5 can be vertically disposed on a desk surface, and when the detection unit 5 is operated, a position on the living body E inserted by the needle D can be correspondingly arranged below the detection unit 5, so that the detection unit 5 is configured to test the section of the tube C adjacent to the needle D.

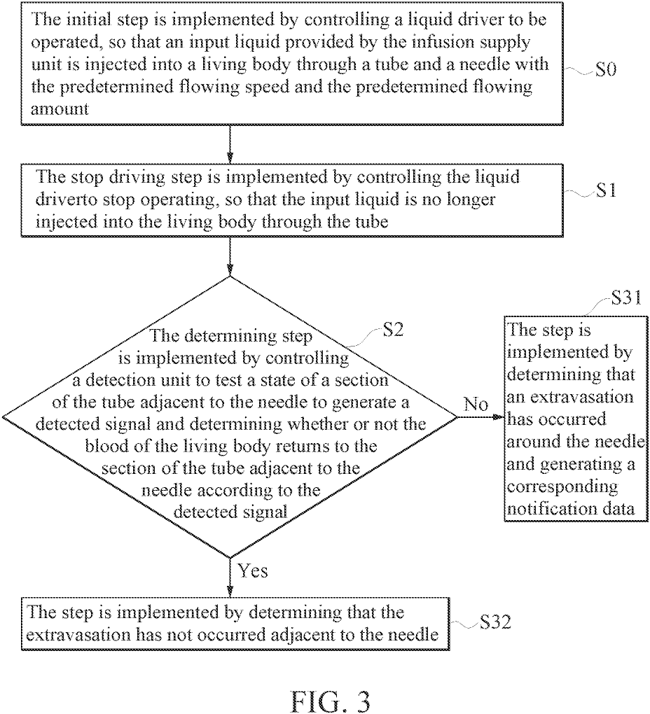

Referring to FIG. 1 to FIG. 3, FIG. 3 is a flowchart of a blood return detecting procedure of the present disclosure. When the needle D is inserted into a blood vessel of the living body E and the infusion apparatus A is turned on, the controller 1 controls the liquid driver 2 to be operated, so that the input liquid B1 provided by the infusion supply unit B is injected into the living body E through the tube C and the needle D with a predetermined flow speed and a predetermined flow amount.

After the input liquid B1 of the infusion supply unit B is injected into the living body E, the controller 1 performs a blood return detecting procedure. In a practical application, the controller 1 can determine that the input liquid B1 is already injected into the living body E by controlling an operating time period of the infusion supply unit B. For example, after the controller 1 controls the infusion supply unit B to be operated for a predetermined time period (e.g., 2 minutes), the blood return detecting procedure is started. In one embodiment, the predetermined time period can be changed by the user.

In other embodiments, the infusion apparatus A further includes a liquid blocking unit to block or reduce a pressure generated by the liquid driver 2 to the input liquid B1 in the tube C. For example, the liquid blocking unit can reduce a potential energy of the input liquid B1 in the tube C adjacent to the needle D that is from the tube C adjacent to the liquid driver 2, so as to accelerate a speed at which the blood of the living body E returns to the section of the tube C adjacent to the needle D, and allow the blood to return to the tube C adjacent to the needle D more easily. The liquid blocking unit can be arranged below the liquid driver 2, or can be arranged at a suitable position between the liquid driver 2 and the needle D. In one embodiment, the liquid blocking unit can be a pressurizing device for pressurizing the tube C to flatten the tube C. In other embodiments, the liquid blocking unit can be an electric water valve, and the controller 1 outputs a controlling signal to control the electric water valve to be operated.

In other embodiments, the controller 1 can determine whether the input liquid B1 is injected into the living body E by detecting an operating state of the infusion supply unit B. For example, the infusion supply unit B can include a motor, and after the controller 1 controls the motor to be operated, the controller 1 synchronously monitors an amount of current of the motor. When the input liquid B1 is injected into the living body E, there will be an apparent change in the amount of current of the motor. At this time, the controller 1 determines that the input liquid B1 is already injected into the living body E, and the blood return detecting procedure is accordingly started. Or, the infusion supply unit B can include a flow speed detector, the controller 1 is electrically connected to the flow speed detector, and the flow speed detector is configured to detect the flow speed of the input liquid B1 in the tube C. When the controller 1 determines through the flow speed detector that the flow speed of the input liquid B1 is decreased, the controller 1 then determines that the input liquid B1 is already injected into the living body E, and the controller 1 starts to perform the blood return detecting procedure.

The blood return detecting procedure includes the following steps.

A stop driving step S1 is implemented by controlling the liquid driver 2 to stop operating to reduce a hydraulic pressure in the tube, so that the input liquid B1 is no longer injected into the living body E through the tube C.

A determining step S2 is implemented by controlling the detection unit 5 to test the state of the section of the tube C adjacent to the needle D to generate the detected signal 51 and determining whether or not the blood of the living body E returns to the section of the tube adjacent to the needle according to the detected signal 51.

If the controller 1 determines that the blood of the living body E does not return to the section of the tube C adjacent to the needle D according to the detected signal 51, a step S31 is implemented by determining that an extravasation has occurred at a periphery of the needle D and generating a corresponding notification data 11.

If the controller 1 determines that the blood of the living body E returns to the section of the tube C adjacent to the needle D according to the detected signal 51, a step S32 is implemented by determining that the extravasation has not occurred adjacent to the needle D and re-operating the liquid driver 2.

It should be noted that, in order to clearly show a procedure of the method for testing extravasation of the present disclosure being applied in a practical application, before the stop driving step S1 in FIG. 3, an initial step S0 is depicted in FIG. 3. The initial step S0 is implemented by controlling the liquid driver 2 to be operated, so that the input liquid B1 provided by the infusion supply unit 2 is injected into the living body E through the tube C and the needle D with the predetermined flow speed and the predetermined flow amount. In other words, before implementing the stop driving step S1 included by the blood return detecting procedure of the present disclosure, the input liquid B1 is required to be injected into the living body E in advance, but the manner in which the input liquid B1 is injected into the living body E can be selected according to practical requirements, and is not limited in the blood return detecting procedure of the present disclosure.

In a practical application, after the controller 1 generates the notification data, the notification data 11 can be transmitted to the monitor 4 to notify the user that the extravasation has occurred at the periphery of the needle D. For example, when the controller 1 determines that the extravasation has occurred at the periphery of the needle D, the user can see related notification text such as “extravasation has occurred” from the monitor 4. Conversely, if the controller 1 determines that the extravasation has not occurred at the periphery of the needle D, the user can see related notification text such as “extravasation has not occurred” from the monitor 4. In other embodiments, the controller 1 can transmit the notification data 11 to an external electronic apparatus (e.g., a smartphone, a tablet, or a remote server) in a wired manner or a wireless manner to notify the relevant personnel.

In a practical embodiment, the detection unit 5 can include a light emitting unit (e.g., a light emitting diode) and a light receiver. In the determining step S2, the controller 1 can control the detection unit 5 to be operated in a wireless manner or a wired manner, so that the light emitting unit emits a light beam toward the section of the tube C adjacent to the needle D, and the light receiver can receive the light beam reflected by the liquid in the tube C and accordingly generate the detected signal 51. Afterwards, the controller 1 can obtain the detected signal 51 in a wireless manner or a wired manner and determine whether or not the blood of the living body E returns to the section of the tube C adjacent to the needle D according to the detected signal 51.

Furthermore, oxyhemoglobin in the blood of the human body generally absorbs infrared light having a wavelength within a range from 850 nm to 1000 nm, and deoxyhemoglobin in the blood generally absorbs red light having a wavelength within a range from 600 nm to 750 nm. Therefore, in a practical application, the light beam can be an infrared light beam or a red light beam. When the tube C has blood therein, most of the red light beam or the infrared light beam emitted by the light emitting unit of the detection unit 5 are absorbed by the hemoglobin in the blood, and the light receiver of the detection unit 5 can only receive the red light beam or the infrared light beam having a relatively low energy. Conversely, when the tube C does not have blood therein, the red light beam or infrared light beam emitted by the light emitting unit of the detection unit 5 are not absorbed, and the light receiver of the detection unit receives the infrared light beam having a relatively high energy.

In a practical application, the light emitting unit and the light receiver of the detection unit 5 can be fixed in a shell. The shell can be connected to a tie that is tied on the living body E, and one portion of the light emitting unit exposed from the shell and one portion of the light receiver exposed from the shell are configured to correspondingly abut against the section of the tube C adjacent to the needle D. In other embodiments, the light emitting unit and the light receiver can be detachably fixed to the section of the tube C adjacent to the needle D, and the light emitting unit and the light receiver are disposed on two opposite sides of the tube C. In other embodiments, the detection unit 5 can include a holding mechanism, and the holding mechanism can fixedly hold on the section of the tube C adjacent to the needle D. When the holding mechanism fixedly holds on the section of the tube C adjacent to the needle D, the light emitting unit and the light receiver face each other.

In other embodiments, the detection unit 5 can include an image capture device. In the determining step S2, the controller 1 can control the image capture device to capture an image of the section of the tube C adjacent to the needle D to generate a captured image (i.e., the detected signal 51), and the controller 1 can then determine whether the extravasation has occurred at the periphery of the needle D according to the captured image. In a practical application, the captured image can be, for example, a color image, but the present disclosure is not limited thereto. The controller 1 can determine whether the tube C has blood therein or not by analyzing the captured image and determining whether a pixel of a corresponding positon in the tube C is red or not. In other embodiments, the detection unit 5 can further include an illuminating unit, and the illuminating unit is configured to illuminate an image capture range of the image capture device, so that the image capture device can clearly capture an image of a region encompassing the periphery of the needle D. Similar to the above descriptions, the image capture device included by the detection unit 5 can be disposed in a shell, the shell can be fixed to the living body E and the tube C through a tie or a holding mechanism, and one portion of the image capture device exposed from the shell is disposed adjacent to the tube C and configured to capture an image in the tube C.

Accordingly, after the input liquid B1 is injected into the living body E, the controller 1 of the infusion apparatus A of the present disclosure can perform the blood return detecting procedure, so as to determine whether the extravasation has occurred at the periphery of the needle D by determining whether the blood of the living body E returns to the section of the tube C adjacent to the needle D. If the controller 1 determines that the extravasation has occurred at the periphery of the needle D, the controller 1 generates the notification data 11 in time, and the relevant personnel can be notified of the extravasation in time. Accordingly, an issue of an inserted portion of the living body E having swelling or skin ulceration due to long-term extravasation can be effectively prevented.

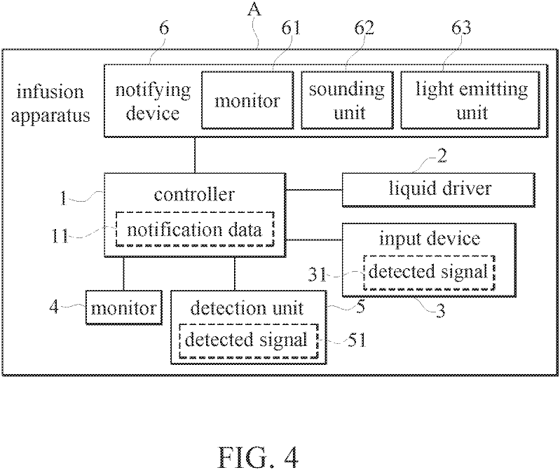

Referring to FIG. 1 and FIG. 4, FIG. 4 is a block diagram of an infusion apparatus according to a second embodiment of the present disclosure. One of the differences between the present embodiment and the previous embodiment is that the infusion apparatus A further includes a notifying device 6 that is electrically connected to the controller 1, and the controller 1 can control the notifying device 6 to be operated.

Another difference between the present embodiment and the previous embodiment is that the input device 3 can generate a test order 31 according to an operation of the user. After the controller 1 receives the test order 31, the controller 1 controls the liquid driver 2 to be operated, so as to inject a test amount (e.g., 10 cc, but the present disclosure is not limited thereto) of the input liquid B1 (e.g., a liquid medicine or a non-medicinal liquid) into the living body E and then perform the blood return detecting procedure. If the controller 1 determines that the extravasation has occurred at the periphery of the needle D, the controller 1 transmits the notification data 11 to the notifying device 6 to notify the user that the extravasation has occurred at the periphery of the needle D. If the controller 1 determines that the extravasation has not occurred at the periphery of the needle D, the controller 1 controls the notifying device 6 to be correspondingly operated, so as to notify the user that the extravasation has not occurred at the periphery of the needle D.

Specifically, the notifying device 6 can include at least one of a monitor 6, a sounding unit 62, and a light emitting unit 63. The input device 3 can include a testing button 3A. After the user (e.g., relevant medical personnel) connects one end of the tube C to the infusion apparatus A, the user further connects another end of the tube C to the needle D, and inserts the needle D into the living body E, then, the user can press the testing button 3A, so that the input device 3 generates the test order 31.

When the controller 1 receives the test order 31, the controller 1 controls the liquid driver 2 to be operated, so that the test amount of the input liquid B1 is injected into the living body E. At this time, the controller 1 further controls the monitor 61 of the notifying device 6 to show relevant notification text, such as “carrying out blood return detecting procedure.” After the test amount of the input liquid B1 is injected into the living body E, the controller 1 proceeds to perform the blood return detecting procedure.

After the controller 1 performs the blood return detecting procedure, if the controller 1 determines that the extravasation has occurred at the periphery of the needle D, the controller 1 controls the monitor 61 of the notifying device 6 to show relevant notification text, such as “extravasation has occurred,” and the controller 1 can further control the sounding unit 62 of the notifying device 6 to produce a corresponding sound and control the light emitting unit 63 of the notifying device 6 to emit corresponding light.

Conversely, if the controller 1 determines that the extravasation has not occurred at the periphery of the needle D, the controller 1 can control the monitor 61 of the notifying device 6 to show relevant notification word, such as “extravasation has not occurred,” and the controller 1 can further control the sounding unit 62 and the light emitting unit 63 of the notification device 6 to be operated, so as to produce a specific sound and emit corresponding light.

According to the above, after the infusion apparatus A, the tube C, and the needle D are mounted together by the user, the user can press the testing button 3A (i.e., operate the input device 3) to allow the controller 1 to have the test amount of the input liquid B1 to be injected into the living body E and the controller then performs the blood return detecting procedure. Afterwards, the user can know whether or not the extravasation has occurred through the operation of at least one of the monitor 61, the sounding unit 62, and the light emitting unit 63 of the notifying device 6. If the extravasation has not occurred, the user can operate the infusion apparatus A to inject the input liquid B1 into the living body E with the predetermined flow speed and the predetermined flow amount. Conversely, the user can be made aware of the occurrence of extravasation, and the user can check and adjust the tube C and the needle D. It is worth mentioning that, in a practical application, the monitor 61 of the notifying device 6 and the above-mentioned monitor 4 can be integrated into a singular monitor.

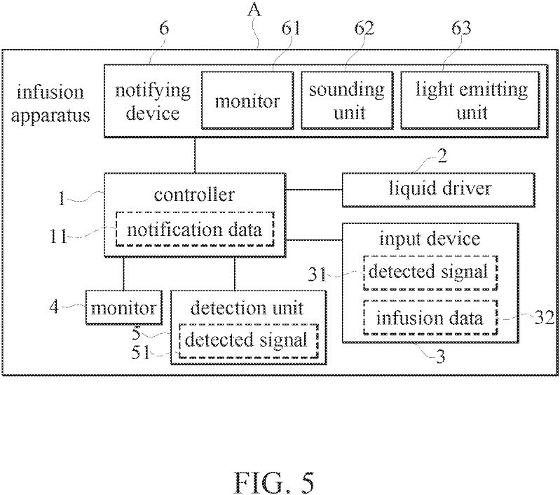

Referring to FIG. 1 and FIG. 5, FIG. 5 is a block diagram of an infusion apparatus according to a third embodiment of the present disclosure. One of the differences between the present embodiment and the above-mentioned second embodiment is that the controller 1 can perform an infusion procedure to inject a predetermined amount of the input liquid B1 into the living body E. Specifically, when the controller 1 performs the infusion procedure, the controller 1 controls the liquid driver 2 to be operated, so as to inject the input liquid B1 provided by the infusion supply unit B into the living body E with a predetermined flow amount and a predetermined flow speed. As a result, the predetermined amount of the input liquid B1 is injected into the living body E.

In a practical application, the input device 3 can correspondingly generate an infusion data 32 according to the operation of the user, and the input device 3 can transmit the infusion data 32 to the controller 1. When the controller 1 receives the infusion data 32, the controller 1 can perform the infusion procedure according to the infusion data 32. For example, the input device 3 can include a plurality of physical buttons, and the controller 1 can control the monitor 4 to show corresponding data according to the operation state of the physical buttons. According to a condition of a patient (i.e., a human being or a pet), the user (e.g., relevant medical personnel) can operate the physical buttons and look at the value shown by the monitor 4 to configure an injection amount per minute, an injection speed per minute, and a total injection amount of the input liquid. After the input device 3 is operated by the user, the input device 3 generates corresponding infusion data 32 (e.g., data including the injection amount per minute of the input liquid, the total injection amount of the input liquid, and the total injection time of the input liquid). The input device 3 then transmits the infusion data 32 to the controller 1, and after the controller 1 receives the infusion data 32, the controller 1 performs the infusion procedure according to the related data included in the infusion data 32.

Another difference between the previous embodiment and the above-mentioned second embodiment is as follows. When the controller 1 receives the test order 31, if the controller 1 is carrying out the infusion procedure, the controller 1 stops carrying out the infusion procedure and performs the blood return detecting procedure. If the controller 1 determines that the extravasation has not occurred at the periphery of the needle D after carrying out the blood return detecting procedure, the controller 1 continues carrying out the infusion procedure and controls the notifying device 6 to be correspondingly operated, so as to notify the user that the extravasation has not occurred at the periphery of the needle D. If the controller 1 determines that the extravasation has occurred at the periphery of the needle D after carrying out the blood return detecting procedure, the controller 1 does not go on to carrying out the infusion procedure and the controller controls the notifying device 6 to be correspondingly operated, so as to notify the user that the extravasation has occurred at the periphery of the needle D.

In brief, in the process where the controller 1 performs the infusion procedure to inject the predetermined amount of the input liquid B1 into the living body E, if the user operates the input device 3 to transmit the test order 31 to the controller 1, the controller 1 stops carrying out the infusion procedure and performs the blood return detecting procedure, and after the controller 1 completes carrying out the blood return detecting procedure, the controller 1 decides whether or not to go on to perform the infusion procedure according to whether the extravasation has occurred at the periphery of the needle D.

For example, after the tube C, the needle D, and the infusion apparatus A are correctly mounted together by the user and the user operates the infusion apparatus A to allow the infusion apparatus A to start injecting the input liquid B1 into the living body E with the predetermined flow amount and the predetermined flow speed. If the user finds that the flow speed of the input liquid B1 in the tube C is different from the predetermined flow speed, the user can operate the input device 3 so that the input device 3 transmits the test order 31 to the controller 1. Accordingly, the controller 1 can pause the infusion procedure and perform the blood return detecting procedure. In this way, the user can confirm whether an occurrence of extravasation causes the flow speed of the input liquid B1 in the tube C to decrease. Conversely, if the controller 1 determines that the extravasation has not occurred at the periphery of the needle D, the user can check whether the tube C is pressed by an external force or not.

According to the above, the infusion apparatus A of the present embodiment enables the user to configure the flow speed and the flow amount of the input liquid B1 by operating the input device 3, and the input liquid B1 is injected into the living body E, so as to generate the infusion data 32. The controller 1 can perform the infusion procedure according to the infusion data 32, and the user can press the testing button 3A at any time according to requirements to control the controller 1 to pause the current infusion procedure and perform the blood return detecting procedure for confirming whether the extravasation has occurred or not.

In a different embodiment, when the controller 1 performs the infusion procedure, the controller 1 can automatically perform the blood return detecting procedure at a predetermined interval (e.g., every 10 minutes), so as to timely determine whether the extravasation has occurred or not.

In a practical application, if the input liquid is a liquid medicine, before the controller 1 performs the infusion procedure, the controller 1 can control the liquid driver 2 to inject a test amount of the liquid medicine into the living body E and the controller 1 can perform the blood return detecting procedure. After the controller 1 performs the blood return detecting procedure, if the controller 1 determines that the extravasation has not occurred at the periphery of the needle D, the controller 1 goes on to perform the blood return detecting procedure to inject a remaining amount of the liquid medicine into the living body E, and the remaining amount is a difference between the test amount and the predetermined amount. If the controller 1 performs the blood return detecting procedure and determines that the extravasation has occurred at the periphery of the needle D, the controller 1 does not go on to perform the infusion procedure, and the controller 1 transmits the notification data 11 to the notifying device 6 to notify the user that the extravasation has occurred at the periphery of the needle D.

Specifically, if the user sets up the infusion apparatus A by operating the input device 3 to inject 300 cc of a liquid medicine into the living body E in 1 hour, after the user turns on the infusion apparatus A, the controller 1 can inject 10 cc of the liquid medicine into the living body E in advance and perform the blood return detecting procedure. If the controller 1 determines that the extravasation has not occurred at the periphery of the needle D, the controller 1 controls the liquid driver 2 to inject the remaining 290 cc of the liquid medicine into the living body E with a predetermined flow amount and a predetermined flow speed as configured by the user. Conversely, if the controller 1 determines that the extravasation has occurred at the periphery of the needle D, the controller 1 does not inject the remaining 290 cc of the liquid medicine into the living body E.

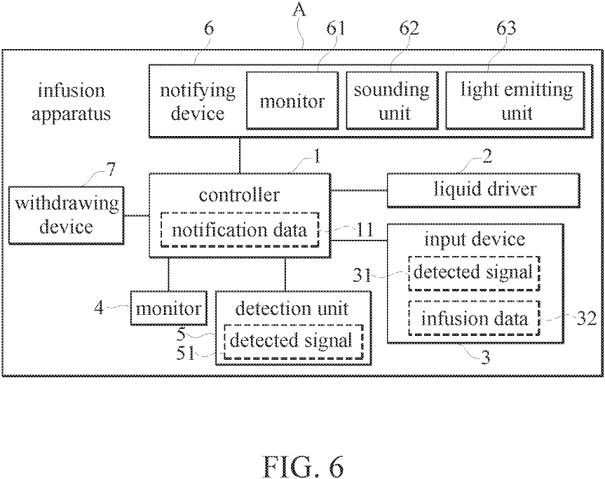

Referring to FIG. 1 and FIG. 6, FIG. 6 is a block diagram of an infusion apparatus according to a fourth embodiment of the present disclosure. The difference between the present embodiment and the above-mentioned third embodiment is as follows. The infusion apparatus A can further include a withdrawing device 7, the withdrawing device 7 is connected to the tube C, and the withdrawing device 7 is electrically connected to the controller 1. After the controller 1 injects the test amount of the liquid medicine (i.e., the above-mentioned input liquid B1) into the living body E and performs the blood return detecting procedure, if the controller 1 determines that the extravasation has occurred at the periphery of the needle D, the controller 1 can control the withdrawing device 7 to be operated, so as to withdraw, through the tube C, the liquid medicine injected into the living body E. In this way, the liquid medicine does not flow out of the blood vessel, and damage to partial tissues of the living body E can be prevented. In addition, the withdrawing device 7 can be an embodiment of the liquid blocking unit, and the withdrawing device 7 can reduce a potential energy of the input liquid B1 in the tube C adjacent to the needle D that is from the tube C adjacent to the liquid driver 2, so as to accelerate the speed at which the blood of the living body E returns to the section of the tube C adjacent to the needle D.

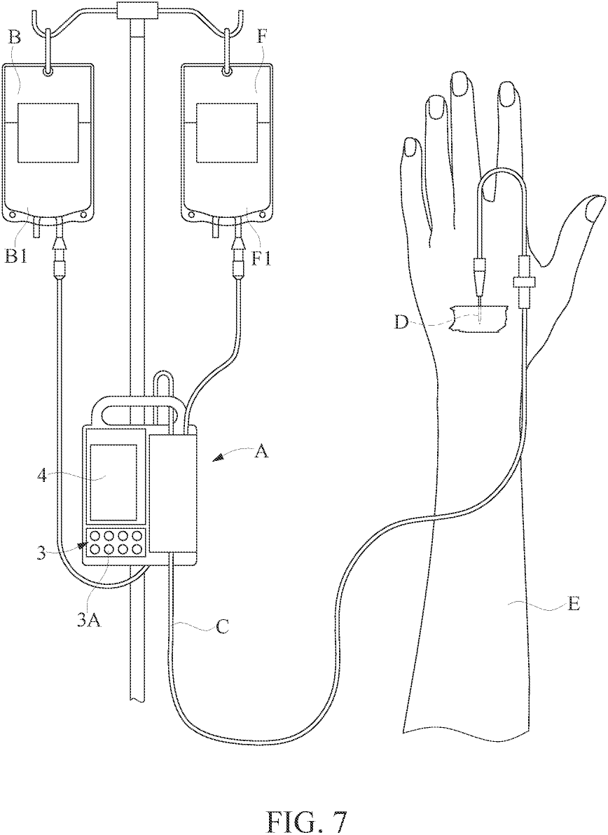

Referring to FIG. 5 and FIG. 7, FIG. 7 is a schematic view of an infusion apparatus according to a fifth embodiment of the present disclosure. The difference between the present embodiment and the above-mentioned third embodiment is as follows. The liquid driver 2 is further connected to an auxiliary liquid providing unit F, and the auxiliary liquid providing unit F is further connected to the tube C. If the controller 1 determines that the extravasation has occurred at the periphery of the needle D, the controller 1 does not perform the infusion procedure, and the controller 1 controls the liquid driver 2 to be operated, so as to inject an auxiliary liquid F1 provided by the auxiliary liquid providing unit F into the living body E. The auxiliary liquid can dilute or neutralize the liquid medicine, or, the auxiliary liquid can help the living body E absorb the liquid medicine.

According to the above, before the controller 1 performs the blood return detecting procedure, the liquid medicine (i.e., the above-mentioned input liquid B1) is injected into the living body E. After the controller 1 performs the blood return detecting procedure, if the controller 1 determines that the extravasation has occurred at the periphery of the needle D, the controller 1 can control the withdrawing device 7 to be operated to withdraw the liquid medicine injected into the living body E, or the controller can control the liquid driver 2 to inject the auxiliary liquid provided by the auxiliary liquid providing unit F into the living body E to dilute the liquid medicine or help the living body E absorb the liquid medicine. In this way, damage from the liquid medicine to the relevant tissue of the living body E due to extravasation can be effectively prevented.

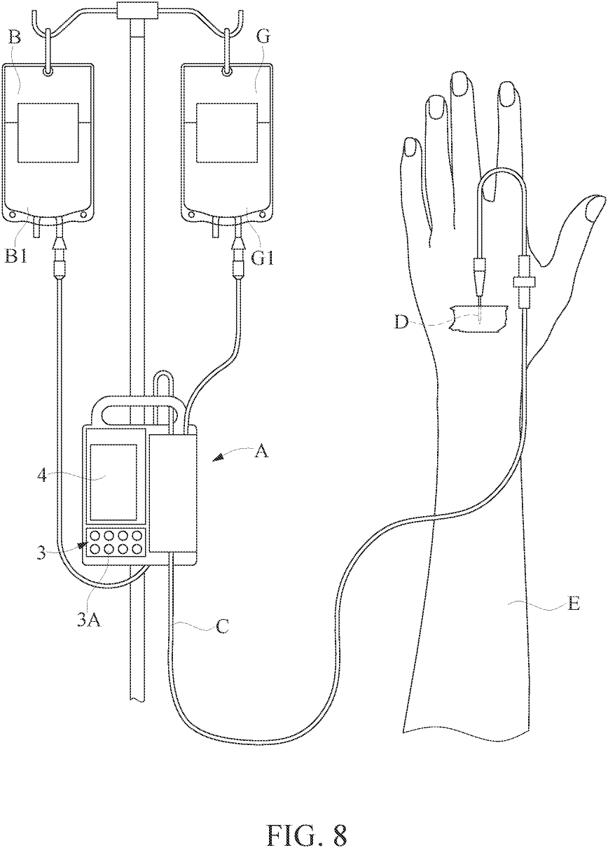

Referring to FIG. 5 and FIG. 8, FIG. 8 is a schematic view of an infusion apparatus according to a sixth embodiment of the present disclosure. The difference between the present embodiment and the previous embodiment is as follows. The liquid driver 2 is further connected to a test liquid providing unit G, and the test liquid providing unit G is connected to the tube C.

When the controller 1 receives the infusion data 32, the controller 1 can control the liquid driver 2 to be operated, so as to inject a test amount of the test liquid G1 into the living body E, and the controller 1 then performs the blood return detecting procedure. After the controller 1 performs the blood return detecting procedure, if the controller 1 determines that the extravasation has not occurred at the periphery of the needle D, the controller 1 injects the predetermined amount of the liquid medicine into the living body E according to the infusion data 32. After the controller 1 performs the blood return detecting procedure, if the controller 1 determines that the extravasation has occurred at the periphery of the needle D, the controller 1 does not perform the infusion procedure, and the controller 1 transmits the notification data 11 to a notifying device 6 to notify the user that the extravasation has occurred at the periphery of the needle D. In other words, before the controller 1 performs the infusion procedure, the controller 1 injects the test liquid that is not harmful to the living body E into the living body E, and the controller 1 then performs the blood return detecting procedure. In this way, even though the controller 1 determines that the extravasation has occurred at the periphery of the needle D after the controller 1 performs the blood return detecting procedure, the living body E is not damaged.

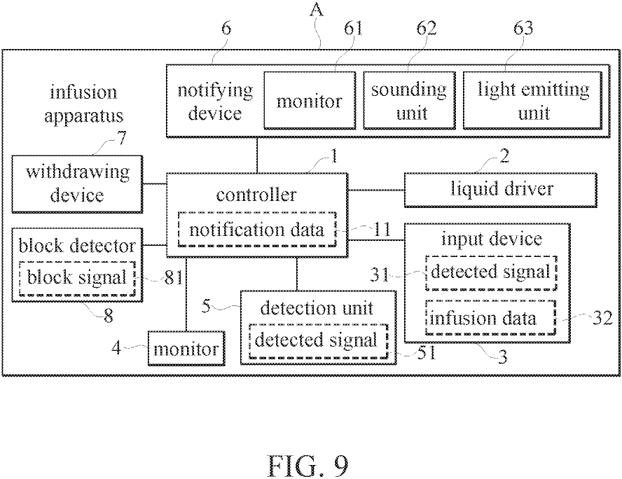

Referring to FIG. 1 and FIG. 9, FIG. 9 is a block diagram of an infusion apparatus according to a seventh embodiment of the present disclosure. As shown in FIG. 1 and FIG. 9, the difference between the present embodiment and the above-mentioned fourth embodiment is that the infusion apparatus A further includes a block detector 8. The block detector 8 is configured to detect a flowing state of the input liquid B1 in the tube C, generate a block signal 81 when the input liquid B1 in the tube C is blocked, and transmit the block signal 81 to the controller 1. For example, the block detector 8 can include a flow speed measuring unit and a processor, the flow speed measuring unit is configured to measure the flow speed of the input liquid B1 in the tube C in time, the processor is electrically connected to the flow speed measuring unit, and the processor can transmit the block signal 81 when the flow speed of the input liquid B1 in the tube C measured by the flow speed measuring unit is apparently decreased.

When the controller 1 performs the infusion procedure, the controller 1 controls the block detector 8 to be operated at the same time, and if the controller 1 receives the block signal 81, the controller 1 stops the infusion procedure and performs the blood return detecting procedure. After the controller 1 performs the blood return detecting procedure, if the controller 1 determines that the extravasation has occurred at the periphery of the needle D, the controller 1 does not perform the infusion procedure, and the controller 1 transmits the notification data 11 to the notifying device 6, so as to notify the user that the extravasation has occurred at the periphery of the needle D. After the controller 1 performs the blood return detecting procedure, if the controller 1 determines that the extravasation has not occurred at the periphery of the needle D, the controller 1 does not perform the infusion procedure, and the controller 1 transmits another notification data to the notifying device 6 to notify the user that the tube C is blocked. For example, the user can see relevant text, such as “extravasation has not occurred, but the tube is blocked” from the monitor 61 of the notifying device 6.

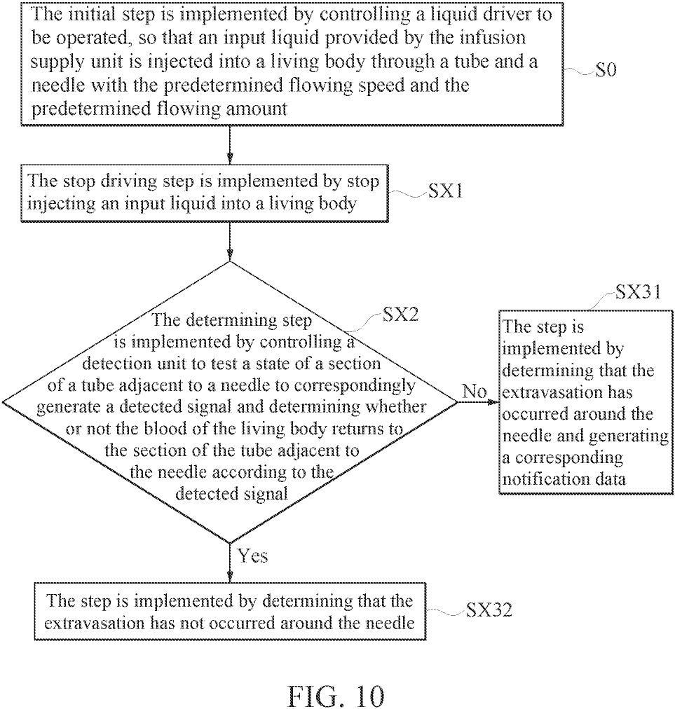

Referring to FIG. 10, FIG. 10 is a flowchart of a method for testing extravasation of the present disclosure. The method for testing extravasation is configured to be performed by a controller of an infusion apparatus. The infusion apparatus is connected to an input liquid providing unit, the infusion apparatus is connected to a tube, and one end of the tube is connected to a needle. The infusion apparatus can inject an input liquid provided by the input liquid providing unit into a living body through the tube and the needle with a predetermined flow speed and a predetermined flow amount. When the infusion apparatus injects the input liquid into the living body, the controller of the infusion apparatus can perform the method for testing extravasation. The practical application of infusion apparatus, the controller, the tube, and the needle of the present embodiment can be referred to in the previous embodiments, and will not be reiterated herein. Naturally, the infusion apparatus of the present embodiment is not limited to being the infusion apparatus of the previous embodiments.

The method for testing extravasation includes the following steps.

A stop driving step SX1 is implemented by stopping injection of an input liquid into a living body.

A determining step SX2 is implemented by controlling a detection unit to test a state of a section of a tube adjacent to a needle to correspondingly generate a detected signal and determining whether the blood of the living body returns to the section of the tube adjacent to the needle according to the detected signal.

If the blood of the living body is determined to return to the section of the tube adjacent to the needle, a step SX31 is implemented by determining that the extravasation has occurred at the periphery of the needle and generating a corresponding notification data.

If the blood of the living body is determined to not return to the section of the tube adjacent to the needle, a step SX32 is implemented by determining that the extravasation has not occurred at the periphery of the needle.

The description of the detection unit of the present embodiment can be referred to in the previous embodiments, and will not be reiterated herein. Naturally, as long as the detection unit can generate the detected signal that enables the controller to determine whether the blood of the living body returning to the tube exists in the section of the tube adjacent to the needle, the detection unit can be different from the detection unit of the previous embodiments.

It should be noted that, in order to clearly show the procedure of the method for testing extravasation of the present disclosure in a practical application, before the stop driving step SX1 in FIG. 10, an initial step S0 is depicted in FIG. 10. The initial step S0 is implemented by controlling the liquid driver to be operated, so that the input liquid provided by the infusion supply unit is injected into the living body through the tube and the needle with a the predetermined flow speed and a predetermined flow amount. In other words, before implementing the stop driving step SX1 included by the method for testing extravasation of the present disclosure, the input liquid B1 is required to be injected into the living body in advance, but the manner in which the input liquid is injected into the living body can be selected according to practical requirements, and the method for testing extravasation of the present disclosure is not limited thereto.

### BENEFICIAL EFFECTS OF THE EMBODIMENT

In conclusion, the infusion apparatus and the method for testing extravasation of the present disclosure can help relevant personnel find and solve the extravasation in time. Accordingly, damage to the living body due to extravasation can be effectively prevented.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An infusion apparatus, comprising:
a liquid driver configured to be connected to at least one infusion supply unit; wherein the liquid driver is connected to one end of a tube, and another end of the tube is connected to a needle;
a detection unit configured to test a state of a section of the tube adjacent to the needle to correspondingly generate a detected signal;
a controller electrically connected to the liquid driver and the detection unit; wherein the controller is configured to control the liquid driver to be operated, so that an input liquid from the at least one infusion supply unit is injected and a blood return detecting procedure is performed by the controller; and wherein the blood return detecting procedure comprises:
controlling the liquid driver to stop operating to reduce a hydraulic pressure in the tube; and
controlling the detection unit to detect the state of the section of the tube adjacent to the needle to generate the detected signal, so as to determine whether or not the blood of the living body returns to the section of the tube adjacent to the needle;
wherein, in response to the blood of the living body not returning to the section of the tube adjacent to the needle, an extravasation is determined to have occurred and a notification data is correspondingly generated;
wherein, in response to determining that the blood returns to the section of the tube adjacent to the needle, the extravasation is determined to not have occurred and the liquid driver is re-operated.

2. The infusion apparatus according to claim 1, wherein the detection unit comprises a light emitting unit and a light receiver, the light emitting unit is configured to emit a light beam toward the section of the tube adjacent to the needle, and the light receiver is configured to receive the light beam from the light emitting unit to generate the detecting signal.

3. The infusion apparatus according to claim 1, wherein the detection unit an image capture device.

4. The infusion apparatus according to claim 1, wherein the controller is configured to perform an infusion procedure to control the liquid driver to inject a predetermined amount of the input liquid into the living body.

5. The infusion apparatus according to claim 1, further comprising a withdrawing device electrically connected to the controller; and wherein, when the controller determines that the extravasation has occurred, the controller controls the withdrawing device to be operated, so that the input liquid injected into the living body is withdrawn through the tube, and wherein the input liquid is a liquid medicine.

6. The infusion apparatus according to claim 1, further comprising an auxiliary liquid providing unit; wherein the liquid driver is further connected to an auxiliary liquid providing unit; wherein, when the controller determines that the extravasation has occurred, the controller controls the liquid driver to be operated to input an auxiliary liquid provided by the auxiliary liquid providing unit into the living body; and wherein the input liquid is a liquid medicine, and the auxiliary liquid is configured to dilute the liquid medicine or promote absorption of the liquid medicine by the living body.

7. The infusion apparatus according to claim 1, wherein the liquid driver is further connected to a test liquid providing unit; wherein the input liquid is a liquid medicine, the test liquid providing unit is configured to provide a test liquid, and the test liquid is different from the liquid medicine; and wherein, after the controller controls the liquid driver to inject a test amount of the test liquid into the living body, the blood return detecting procedure is performed by the controller.

8. The infusion apparatus according to claim 1, further comprising a block detector; wherein the block detector is configured to detect a flowing state of the input liquid in the tube; and wherein, when the input liquid in the tube is blocked, the block detector is configured to generate a block signal to the controller.

9. The infusion apparatus according to claim 1, further comprising a liquid blocking unit; wherein the liquid blocking unit is configured to reduce the hydraulic pressure in the tube, so as to enhance the situation of blood returning to the section of the tube adjacent to the needle.

10. A method for detecting extravasation applied in an infusion apparatus, wherein the infusion apparatus is connected to a tube, and one end of the tube is connected to a needle, the method comprising:
stop injecting an input liquid from at least one infusion supply unit to reduce a hydraulic pressure in the tube; and
detecting a state of a section of the tube adjacent to the needle to correspondingly generate a detecting signal and determining whether or not the blood of the living body returns to the section of the tube adjacent to the needle according to the detecting signal;
in response to determining that the blood does not return to the section of the tube adjacent to the needle, the extravasation is determined to have occurred and a notification data is correspondingly generated; and
in response to determining that the blood returns to the section of the tube adjacent to the needle, the extravasation is determined to not have occurred and the liquid driver is re-operated.

11. The method according to claim 10, wherein the infusion apparatus comprises a detection unit comprising a light emitting unit and a light receiver; and wherein, the light emitting unit is configured to emit a light beam toward the section of the tube adjacent to the needle, and the light receiver is configured to receive the light beam and thereby generate the detected signal.

12. The method according to claim 10, wherein the detection unit comprises an image capture device.

13. The method according to claim 10, wherein the input liquid is a liquid medicine, and the infusion apparatus further comprises a withdrawing device electrically connected to the controller; and wherein, in response to the controller determining that the extravasation has occurred, the controller controls the withdrawing device to be operated, so that the liquid medicine is withdrawn through the tube.

14. The method according to claim 10, wherein the input liquid is a liquid medicine, a liquid driver is connected to an auxiliary liquid providing unit; wherein, when the controller determines that the extravasation has occurred, the controller controls the liquid driver to be operated to input an auxiliary liquid provided by the auxiliary liquid providing unit into the living body; and wherein the auxiliary liquid is configured to dilute the liquid medicine or promote absorption of the liquid medicine by the living body.

15. The method according to claim 10, wherein a liquid driver is connected to a test liquid providing unit; wherein the input liquid is a liquid medicine, the test liquid providing unit is configured to provide a test liquid, and the test liquid is different from the liquid medicine; and wherein, after the controller controls the liquid driver to inject a test amount of the test liquid into the living body, the method is performed.

16. The method according to claim 10, wherein the infusion apparatus comprises a block detector; wherein the block detector is configured to detect a flowing state of the input liquid in the tube; and wherein, when the input liquid in the tube is blocked, the block detector is configured to generate a block signal to the controller.

17. The method according to claim 10, wherein the infusion apparatus comprises a liquid blocking unit; wherein the liquid blocking unit is configured to reduce the hydraulic pressure in the tube, so as to enhance the situation of blood returning.
